(19) United States
(12) Patent Application Publication
(10) Pub. No.: US 2019/0076195 A1
(43) Pub. Date: Mar. 14, 2019

(54) ARTICULATING LASER INCISION INDICATION SYSTEM

(71) Applicant: THINK SURGICAL, INC., Fremont, CA (US)

(72) Inventors: Stan G. SHALAYEV, Fremont, CA (US); Joel ZUHARS, Fremont, CA (US)

(21) Appl. No.: 15/767,254

(22) PCT Filed: Sep. 23, 2016
(86) PCT No.: PCT/US2016/053251

§ 371 Date: Apr. 10, 2018

Related U.S. Application Data
(60) Provisional application No. 62/253,968, filed on Nov. 11, 2015.

(51) Int. Cl. A61B 34/10; A61B 18/20; A61B 5/055; A61B 5/00; G06T 7/00; A61B 6/03; A61B 90/13; A61B 90/00

(52) U.S. Cl. CPC A61B 34/10; A61B 18/203; A61B 5/055; A61B 5/0035; A61B 2018/00601; A61B 6/032; A61B 90/13; A61B 90/36; G06T 7/0012

(57) ABSTRACT

A method of planning a minimally invasive surgical incision in a subject includes an image data set of an anatomical region of the subject being received. A surgical plan is created within the image data set. The anatomical region is registered to the surgical plan. A laser is articulated to project a light indication on the subject indicative of a depth of a first incision. A system for implementing the surgical plan on a subject includes a tracking array. A processor receives an initial positional input from the tracking array, three-dimensional scan data of a surgical field and includes software for generating a surgical plan. A laser is positioned to project a light indication on the subject. A controller controls the laser to modify the light indications to indicate a preselected path and a preselected depth of incision. The articulating device receives the position input and the surgical plan.

### FIELD OF THE INVENTION

The present invention generally relates to the field of computer-assisted surgery and in particular, to a new and useful method and system for indicating an incision path based on the depth of the incision.

### BACKGROUND

In any type of surgery, an incision is made to expose an area of interest. Depending on the type of procedure, there may be different incision paths with various advantages and disadvantages. In general, a smaller incision and less tension on the skin results in a faster healing time and reduced tissue trauma to the living subject. At the same time, the incision must be large enough to provide adequate access to the surgical site to perform the procedure.

In total knee arthroplasty, there are a plurality of incision paths a surgeon may create to expose the knee joint. The surgeon must have access to both the distal femur and proximal tibia to create the bone cuts to receive the artificial implants. The characteristics of the subject, such as body mass index (BMI), flexibility, and muscularity all have an impact on the amount of tissue that must be incised to expose the joint area appropriately. In any surgical situation however, if the surgeon is able to minimize the length of the incision or spare the incision of underlying tissues such as muscles or tendons, then the subject is likely to recover faster and with fewer complications.

To aid the surgeon in guiding a preferred or optimized incision path, methods of using lasers or projectors have been proposed to indicate where the underlying target regions are located on the surface of the skin. A major problem with these systems however, is the inability to account for the depth of the incision. In cases where a surgeon must cut through multiple tissue layers, displaying the target region on the skin is insufficient and may lead the incision on a strayed path given the relative movement between the soft tissues and the target region.

Furthermore, the surgeon is unable to plan the preferred path as a function of tissue depth and subsequently receive indicator feedback throughout the incision. By providing the surgeon with the ability to plan a subject specific incision path based on tissue depth, not only can the size of the incision be optimized, but other tissues below the skin can be accounted for throughout the execution of the incision.

Additionally, there are instances when a surgeon may be creating an incision but is unaware of underlying tissue types and subject specific anatomical variants such as bifurcated vessels, nerve positions, or anatomical landmarks. Occasionally, the surgeon may try to locate a certain anatomical landmark beneath the skin to help guide the incision along a desired path, or a path that navigates around the anatomical landmark. That anatomical landmark may be a critical blood vessel or nerve that must be avoided. Since the surgeon can only see the surgical site as it is currently exposed, there is often no indication of how deep these critical regions or anatomical landmarks lie within the surgical site. If a surgeon is relying on these landmarks to help guide the incision, being unaware of their location with respect to the incision makes every additional cut hazardous.

Furthermore, by combining the precision of computer-assisted surgical devices with a laser indication system based on depth, a minimally invasive robotic procedure may be performed to spare soft tissue while repeatedly and accurately executing a procedure on the target region. Having the ability to plan an incision path, plan the surgical procedure and then execute both the preferred incision path and surgical procedure in the operating room is highly advantageous to both the subject and the surgeon.

Thus, there is a need for a laser indication system to guide a surgeon during an incision as a function of tissue depth to reach a target area to minimize unintended tissue exposure. There is a further need for an indication system that provides a real-time indication of upcoming or surrounding tissues/structures before a surgeon performs a subsequent incision. There is an even further need to cohesively combine the use of a laser indication system with other computer-assisted surgical devices to optimize a procedure that results in faster subject recovery times, shorter operating times, and fewer perioperative complications.

### SUMMARY OF THE INVENTION

A method of planning a minimally invasive surgical incision in a subject includes an image data set of an anatomical region of the subject being received. A surgical plan is created within the image data set. The anatomical region is registered to the surgical plan. A laser is articulated to project a light indication on the subject indicative of a depth of a first incision.

A system for implementing the surgical plan on a subject includes a tracking array.

A processor receives an initial positional input from the tracking array, three-dimensional scan data of a surgical field and includes software for generating a surgical plan. A laser is positioned to project a light indication on the subject. A controller controls the laser to modify the light indications to indicate a preselected path and a preselected depth of incision. The articulating device receives the position input and the surgical plan.

### BRIEF DESCRIPTION OF THE DRAWINGS

The present invention is further detailed with respect to the following drawings. These figures are not intended to limit the scope of the present invention but rather illustrate certain attributes thereof.

FIG. 1 illustrates a sagittal image slice from an image data set that may be used to create a surgical plan of a human knee;

FIG. 2 depicts an operating room with a tracking system and articulating lasers to aid in a guiding a surgical incision;

FIGS. 3A-3D illustrate various indications provided by the articulating lasers to help guide a surgical incision; wherein FIG. 3A shows the exterior surface of a subject's knee with lasers delineating a single focused point at the start of an incision path, FIG. 3B depicts laser light to outline an incision path projected on the subject, FIG. 3C depicts laser light projecting an image outlining specific tissue or areas under the exterior surface of the subject;

FIG. 3D depicts laser light projecting an image of text or character string on the subject;

FIG. 4 depicts an operating room with a tracking system, articulating lasers, and a laser depth sensor to aid in guiding a surgical incision; and

FIGS. 5A-5C are schematic illustrating the progression of an incision using one or more modulated pulsed articulating lasers from intact tissue (FIG. 5A) through a partial incision to incomplete depth (FIG. 5B), to a full length incision with a segment being to the correct depth (FIG. 5C).

### DETAILED DESCRIPTION OF THE PREFERRED EMBODIMENTS

The present invention has utility as a system and method for indicating an incision path as a function of tissue depth during a surgical operation. As such, the present invention in simplest form of method leads to the acquisition of data in the form of a laser light image that makes the position and depth of an optimal surgical incision. When used in this simplest manner, an inventive method produces image scans with light which in real time and without undertaking any further steps except for purely mental acts, enable a surgeon to decide on the course of incisive action to be taken. Embodiments of the inventive method and system indicate the surgical path using one or more articulating lasers in association with a tracking system to project the incision path either on or adjacent to the surgical site. The indicator(s) update in real-time based on a measured depth within the incision, a tissue layer and/or the pre-operative plan to create an optimal incision for a given procedure. In some inventive embodiments, the system and method are actually used to make the incisions during a surgical procedure. The surgical procedure so conducted is performed for financial remuneration and therefore constitutes a business method.

The following description of the preferred embodiments of the invention is not intended to limit the invention to these preferred embodiments, but rather to enable any person skilled in the art to make and use this invention. The invention described herein illustratively uses total knee arthroplasty (TKA) as an example. Although total knee arthroplasty is one procedure that may benefit from the disclosed embodiments other surgical procedures that may benefit illustratively include surgery to the hip joint, spine, shoulder joint, elbow joint, ankle joint, jaw, a tumor site, joints of the hand or foot, and other appropriate surgical sites. It should also become apparent to those skilled in the art that the invention disclosed herein may be used in all types of surgical applications such as trauma, orthopedics, neurology, ENT, oncology, podiatry, cardiology and the like. Additionally, use of the present invention in micro-surgical procedures, remote surgical procedures, and robotically controlled incisions is also contemplated.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to included 1-2, 1-3, 2-3, 2-4, 3-4, and 1-4. Additionally, the use of “or” is intended to be inclusive, that is “and/or”, unless stated otherwise.

As used herein, the term “subject” is used to refer to a human, a non-human primate, a cadaver, an animal of a horse, pig, goat, sheep, cow, mouse, or rat.

As used herein, the term “communication” is used to refer to the sending or receiving of data, current or energy through a wired or wireless connection unless otherwise specified. Such “communication” may be accomplished by means well known in the art such as Ethernet cables, BUS cables, Wi-Fi, Bluetooth, WLAN, and the like. The “communication” may also be accomplished using targeted visible light as described in U.S. Prov. Pat. App. Nos. 62/083,052 and 62/111,016 assigned to the assignee of the present application.

As used herein, a fiducial marker refers to a point of reference capable of detection. Examples of a fiducial marker may include: an active transmitter, such as a light emitting diode (LED) or other electromagnetic emitter; a passive reflector, such as a plastic sphere with a retro-reflective film; a distinct pattern or sequence of shapes, lines or other characters; acoustic emitters or reflectors; magnetic emitters or reflectors; radiopaque markers; and the like or any combination thereof. A tracking array is an arrangement of a plurality of fiducial markers in/on a rigid body of any geometric shape, where each tracking array has a unique geometry of fiducial markers or a unique blinking frequency if active LEDs are used to distinguish between different objects.

Disclosed herein is the use of a tracking system. Tracking systems generally include one or more receivers to detect one or more fiducial markers in three-dimensional (3-D) space. The receiver(s) are in communication with at least one processor or computer for processing the receiver output. The processor calculates the position and orientation (POSE) of the one or more fiducial markers and any objects fixed thereto using various algorithms such as time-of-flight, triangulation, transformation, registration or calibration algorithms Examples of tracking systems to determine the POSE of an object are described in U.S. Pat. Nos. 5,282,770, 6,061,644, and 7,302,288. Examples of mechanical tracking systems are described in U.S. Pat. No. 6,322,567. Methods of registration, including imaging registration, and calibration to define an object's coordinate frame with respect to another as well as methods to track objects with tracking arrays, fiducial markers, or mechanically tracked probes attached thereto are known in the art and are described in U.S. Pat. Nos. 5,772,594, 5,951,475, 6,033,415, 6,470,207, 7,043,961, 7,689,019, 8,036,441 and U.S. Pat. App. No. 20140187955.

Embodiments of the present invention include a system and method for creating a minimally invasive incision path. An image data set of an anatomical region of a subject is collected and communicated to a processor. The surgical plan is created by computer software executed by the processor or another processor. The anatomical region from the image data set is registered with the surgical plan. A laser is articulated to project light as an indication on the surgical area of the subject; the indication is representative of a calculated incision path and depth. In more specific embodiments of the inventive method, a first incision is created on the subject, and then a depth of the first incision is measured to yield a measured depth. A signal is provided prior to creating a second incision, where the signal is based on the measured depth and the surgical plan.

As a business method, the subject or a medical insurance entity pays a fee for the above method computation, alone or in combination with the surgical procedure based on the indication of an incisional sequence.

In a specific inventive embodiment the receiving of an image of an anatomical region of a subject is performed pre-operatively or intra-operatively. Image data sets may be collected using an imaging modality illustratively including magnetic resonance imaging (MRI), computed tomography (CT), x-rays, ultrasound, fluoroscopy, and/or combinations thereof commonly referred to as fused or merged image data sets. In a particular embodiment, a three-dimensional (3-D) model is created from the image data set(s) on a computer with imaging software that specifies or separates each tissue layer. The tissue layers may be separated in the image data set(s) using segmentation techniques known in the art such as those described in the article by ChristianTeich in. Tissue Differentiating Segmentation for a Region Anaesthetic Simulation. Inst. Für Med. Informatik. 2007. The 2-D image data sets or 3-D models are used to create a surgical plan. In a particular embodiment, the image data set(s) are captured with the anatomical region in the same position and orientation (POSE) as the procedure is performed. Additionally, a plurality of image data set(s) may be collected with the anatomical region in multiple POSEs.

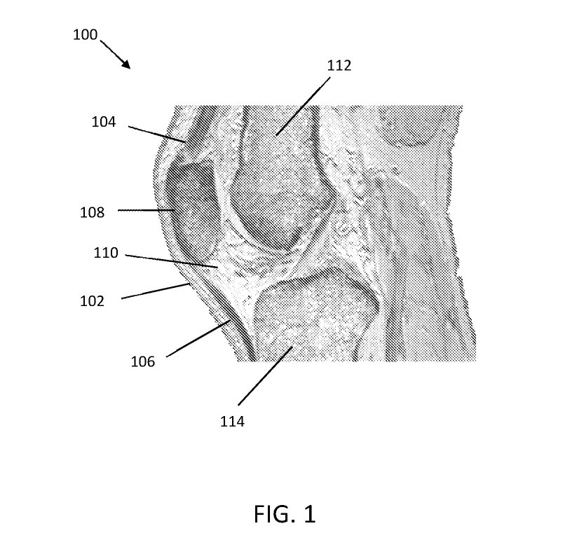

In specific embodiments, the creation of a surgical plan is performed by a user such as a surgeon on a computer with imaging or planning software. On the 2-D image data set(s) or 3-D model, the surgeon may segment, identify, measure or label each soft tissue layer that is of importance to a desired incision path. For example, with respect to FIG. 1, an image slice 100 of a subject's knee in a sagittal view is generally shown. A desired incision path may be directed through some of the tissues shown in FIG. 1. The particular image slice 100 shown may be targeted by the surgeon scrolling through the 2-D image slices in different planar views, such as coronal, sagittal, or axial views, to identify the anatomical region shown at image slice 100 in 3-D space. The surgeon may then highlight, identify, measure, or label the corresponding tissues that appear in the image slice 100. For example, the surgeon may measure the thickness of the skin 102, quadriceps femoris tendon 104, and patellar tendon 106. The surgeon may segment or highlight the patella 108, the fat pad 110, the femur 112, or the tibia 114. In a particular embodiment, each of the tissues of FIG. 1 may be measured relative to other tissues. For example, the distance, torsion, or combination thereof between the proximal portion of the patella 108 and the anterior surface of the femur 112 may be measured.

If 2-D image slices are used to plan the case, the surgeon may go through each slice repeating the above procedure. In some inventive embodiments, the imaging or planning software automatically segments, highlights, measures the thickness or volume of the tissues, or measures the relative distance between each of the different tissues.

In a particular embodiment, a user may virtually perform the incision path on the image data set(s) or 3-D model. The planning software may include a tool to scroll through or make visible each of the tissue layers or anatomical structures in the virtual view. For example, there may be a set of checkboxes in the virtual view, where each checkbox corresponds to a tissue layer or specific anatomical structure. The user may view a particular tissue layer or anatomical structure by checking or unchecking the corresponding checkboxes. With the exterior surface of the anatomy (i.e. skin) in view, the user may define a desired incision path by generating or defining a line or outline on the exterior surface. This exterior incision path may be projected through each of the tissue layers in the planning software. The projected incision path may extend from the most anterior portion of the anatomy to the most posterior portion of the anatomy. The user can then adjust the incision path as a function of tissue depth. For instance, the surgeon may unclick the exterior surface view, to view a next tissue layer, such as the underlying fascia, retinaculum, patella, tendons, muscles, and combinations thereof. The user can then plan, for example, a mini-sub vastus incision 207, to be created on this next tissue layer. The user may repeat the procedure for each tissue layer. It is of note that while the prior art process of drawing an incision line on the subject skin with a pen or marker can delineate an initial incision surgical plan, no information is conveyed as to subsequent cuts or depth limits of such incisions.

The surgical plan may also include instructions for performing a surgical procedure on the targeted anatomical region. The targeted anatomical region refers to the anatomy requiring surgical attention for a surgical procedure. For example, the targeted anatomical region may include: the knee, requiring a total or partial knee arthroplasty; the hip, requiring a total hip arthroplasty; the spine, requiring a spinal fusion or disc replacement; and the like. The surgical plan thus can also include, for example, the POSE of the bone cuts required to implant knee components to restore the mechanical axis of a subject's leg in a total knee arthroplasty procedure. This surgical plan may be uploaded to a computer-assisted surgical device that may aid with creating or guiding the required bone cuts. Well-known systems in the art for aiding in planning and executing the bone cuts to receive implant components are The ROBODOC® Surgical System (THINK Surgical Inc., Fremont, Calif.) and the RIO® Interactive Orthopedic System (Stryker Mako, Ft. Lauderdale, Fla.). It is noted that while embodiments of the present invention may implicitly involve manual surgery, it is appreciated that the present invention is equally suitable for microsurgery procedures performed with the aid of a stereomicroscope or other microsurgical viewing system.

In a particular embodiment, the incision path and the implant placement are cohesively planned such that the working end of a computer-assisted device can access the targeted region through a smaller incision and still be capable of executing the plan.

The final surgical plan is saved for use in the operating room. The surgical plan may also be created intra-operatively on-the-fly using ultrasound or fluoroscopy as further described below. The surgical plan may include any of the embodiments, or combinations thereof, described above including, but not limited to, the labelled anatomy, 3-D bone models, relative distances of the tissues from a rigid tissue such as bone, a volumetric representation of each tissue, specified regions within the tissue relative to other tissues, a set of instructions to be performed on the targeted anatomical region, or a desired incision path throughout each of the tissue layers in three dimensions.

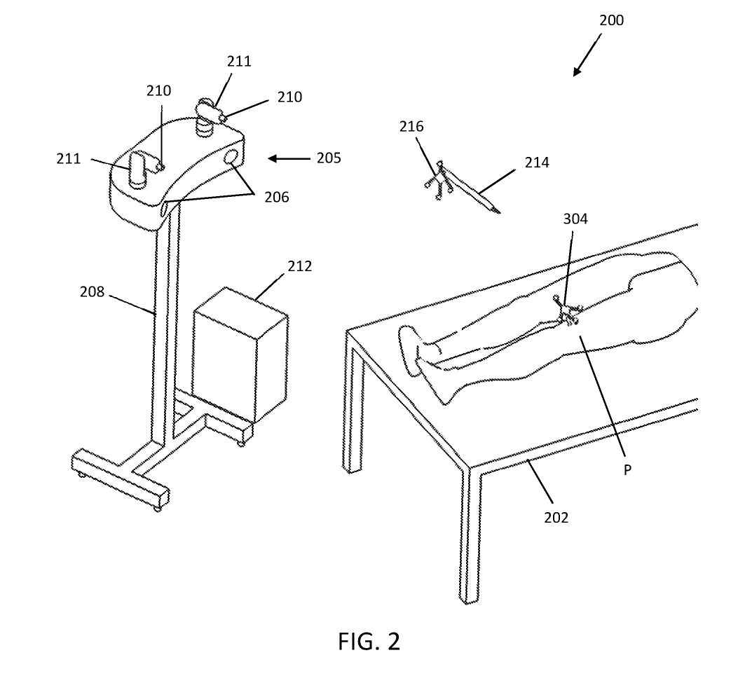

The method of registering the surgical plan to the anatomical region is best described with respect to FIG. 2, which illustrates an operating room (OR) shown generally at 200. A subject P is prepared on a surgical table 202 for a surgical procedure. A bone tracking array 204 is fixed to the operative bone(s) through a small incision made on the subject's skin. The bone tracking array 204 may also be fixed to the bone through a percutaneous puncture. Once the tracking array is fixed, the surgical plan is registered to the bone using techniques known in the art such as those described above. In a particular embodiment, the subject's operating region may be externally fixed to reduce the movement of the bone during the procedure.

The operative bone is tracked to periodically or continuously update the absolute or relative POSE of the bone if the bone moves during the procedure. The tracking system 205 may include two optical receivers 206 in communication with a tracking system computer 212. The tracking system 205 may be located at various locations in the operating room 200 generally above or to the side of the surgical field, with localized/extended surgical field coverage. For example, the tracking system 205 may be attached to the operating room (OR) lights, built into the OR lights, on a boom for an OR light, or on a stand-alone pole system 208.

The step of articulating a laser to project an indication on said subject includes the use of one or more articulated lasers 210 present in the OR 200. The articulated lasers 210 may be for example a single continuous source laser pointer, a line laser, a modulated pulsed laser, or a pico-projector. The articulated lasers 210 may be articulated in one or more degrees of freedom, particularly in two degrees of freedom, by motors or other types of actuators attached by linkages 211. The motors or actuators adjust the POSE of the lasers using surgical plan data and the tracking data from the tracking system 205, through a wired or wireless connection. The motors, actuators, or modulation of one or more pulsed lasers may be controlled by a controller in communication with one of the computers described above or a separate processor.

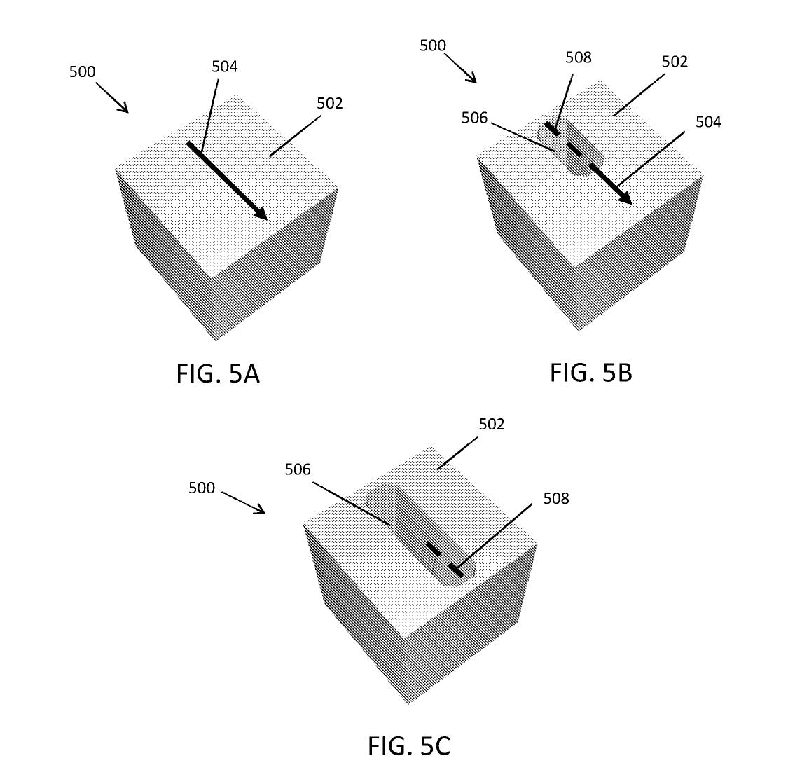

The articulated lasers 210 may be located at various locations in the OR including the OR lights, built into the OR lights, on a boom for an OR light, on a stand-alone pole system 208, attached to a surgical table, attached directly to the subjects anatomy, attached to a tracked object such as a surgical tool or robotic tool, or any other appropriate fixturing point. The articulated lasers 210 may be fixed in a known POSE relative to the tracking system 205. In a particular embodiment, a laser tracking array (213a, 213b) is attached to the articulated lasers 210 to be tracked by the tracking system 205 as depicted in FIG. 4. Having the articulated lasers fixed relative to the tracking system coordinates however, may reduce the computational time that would otherwise be required to continuously update the POSE of the articulated lasers 210 as they move in 3-D space.

Additionally, to ensure the articulated lasers 210 accurately project, highlight, mark, indicate, or identify a specific location in 3-D space, the articulated lasers are calibrated with respect to the tracking system 205. The calibration may be verified intra-operatively by placing a tracked calibration object at one or more POSEs in space that corresponds to a known focal point between two or more articulated lasers in a known POSE. If the lasers converge at the focal point on the tracked calibration object at one or more POSEs, then the calibration is verified. A similar procedure may be performed pre-operatively with multiple calibration objects in multiple POSEs to increase accuracy.

In a specific embodiment, the laser indication system may be used with a computer-assisted robotic device to calibrate or verify the calibration of a dynamic (i.e., articulated) or static laser system. Such robotic devices are disclosed in U.S. Pat. Nos. 5,086,401 and 7,206,626 which are incorporated by reference herein in its entirety. Multiple lasers, particularly but not limited to three lasers, in a static calibrated configuration with a known focal point, may be used with a robot, where the robot positions an object at the focal point. The robot positions an object at the focal point, given that the robot is tracked and receives the tracking data from the tracking system 205, such that the robot knows the focal position within a coordinate space also known by the tracking system 205 and relative to subject positioning in the operating field. In this case, if the focal spot is seen by the tracking system 205 or a viewing camera to be of a known configuration, then it will be demonstrated that the robot and tracking system 205 are well calibrated relative to each other, with continuous verification. If the focal spot is different than the expected size, or the shape is not, for example, circular, the images from the tracking system cameras 206 can be used to compute the correct position for the robot to move to perfect the precise position of the focal spot. Therefore, the robot can have its calibration perfected immediately in real time. If the lasers are of different colors, such as red, yellow, and blue, then the color and shape of the spot should be as expected, for simplified verification and confirmation, for example a white circular spot of a certain radius, and a non-white or partially white spot with colored sides may be used also to correct the calibration of the robot or other positioning device. In other instances a summation spot color indicates alignment of light outputs. For example, convergence of yellow and blue light spots produces a green spot to provide a visual projection onto the subject of calibration or other information to the surgeon.

With the articulated laser(s) 210 accurately calibrated with respect to the tracking system coordinates, and the anatomical region registered to the surgical plan in tracking system coordinates, the laser(s) are articulated to project a light indication on the exterior surface of the subject's anatomy according to the surgical plan. In a specific embodiment, the POSE of the exterior surface of the subject's anatomy is known from the surgical plan. Typically, the bone is used as the registration structure because it is rigid and radiopaque. The surgical plan can therefore contain the relative positions or distances of the other tissues, including the exterior surface of the subject, with respect to the surface of the bone as described above. In another embodiment, a depth sensor may be used to mark, measure, or outline, the exterior surface of the subject's anatomy or intra-operative images with fluoroscopy or ultrasound are used to identify the exterior surface to the tracking system 205. The use of a depth sensor and intra-operative images are further described below.

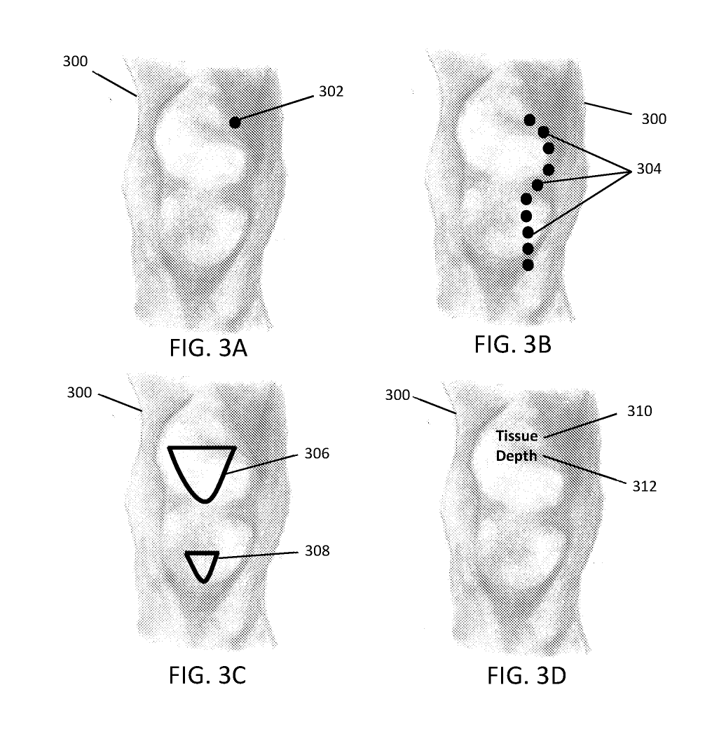

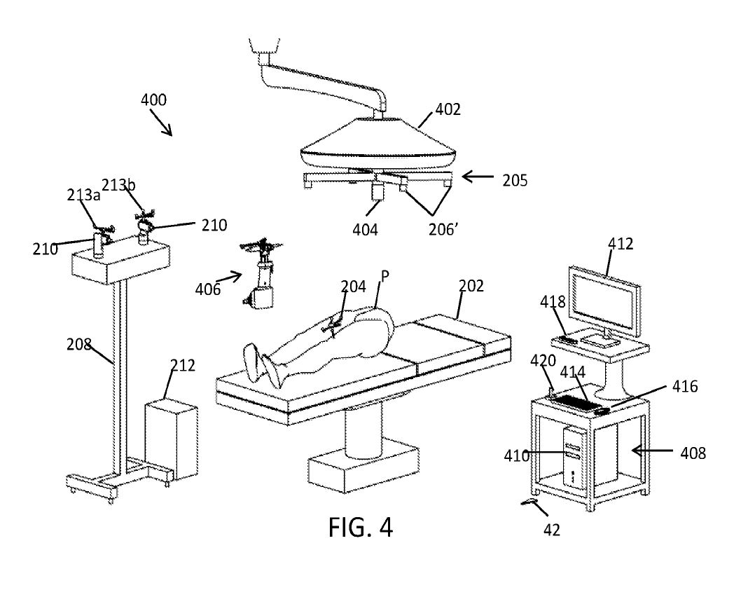

The articulated laser(s) 210 may provide many different types of indications. In a particular embodiment, with reference to FIG. 3A, the exterior surface of a subject's knee is shown at 300. Two or more lasers 210 are articulated to project a single focused point 302 at the start of an incision path designated in the surgical plan. In another embodiment, with reference to FIG. 3B, one or more laser(s) are articulated such that an outline of the incision path 304 is projected on the subject. The laser(s) 210 may be a single point laser and rastered to continuously draw the planned incision path 304. By modulating light intensity along the incision path, information is communicated to the surgeon as to not only the lateral incision path, but also in some embodiments the orthogonal depth of the incision based on the plan. The laser 210 may also be a pico-projector, which projects an image of the planned incision path 304. In a specific embodiment, with reference to FIG. 3C, the articulated lasers 210 may draw or project an image outlining specific tissue or areas under the exterior surface of the subject. For example, the laser(s) 210 may outline the location of the patella 306 and the tibial tuberosity 308. By outlining the tissues or areas, the surgeon can properly gauge where to start an incision, or where to avoid any critical anatomical landmarks or tissues under the visual tissue layer. In a specific embodiment, with reference to FIG. 3D, the laser(s) 210 may project an image of indicia such as text or character string displaying a type of tissue 310 and how deep 312 that type of tissue is from the exterior surface of the subject. For example, the laser(s) 210 may project an image of text “Patellar Tendon” as the tissue 310, and provide a depth 312 of “5 mm”. The depth 312 may update as the surgeon is incising the tissue based on an incision depth measurement from a depth sensor described below. The surgeon may also change what type of tissue 310 is displayed on the subject's skin. Through a voice command, a controller, joystick, or other input device, the surgeon may change the tissue type from, for example, “Patellar Tendon” to “Fat Pad”. The depth 312 would change accordingly to the actual depth of the fat pad.

In a specific embodiment, in spine surgery, the lasers 210 may be articulated to display an image or indicia on top of, or adjacent to the actual vertebrae, such as C1-C2, L1, L2, and also, particular anatomy can be highlighted, such as the entry point position for a pedicle screw.

The step of creating a first incision on the subject includes the use of an incision device. The incision device may be for example a scalpel, lancet, probe, electrocautery device, a hydro-dissection device or any other device used to incise hard or soft tissue in a surgical procedure. In a specific embodiment, the incision device is operated by a computer-assisted surgical device 406 as shown in FIG. 4, illustratively including the devices disclosed in PCT App. Num. US2015/051713 and U.S. Patent Application Publication 2013/0060278. The operation of the incision device by the computer-assisted device can act to provide active, haptic, or passive guidance in creating the incision. Having both guidance from the articulated lasers 210 and the guidance from the computer-assisted device may greatly increase the accuracy of a planned incision. In particular circumstances, where the targeted anatomical region is deep within the tissue layers, and the avoidance of a particular anatomy along the incision path is critical, the dual functionality provides mental security to the surgeon and a better outcome for the subject.

In specific embodiments of the step of measuring the depth of a first incision is accomplished using a depth sensor. In a specific embodiment, with reference to FIG. 2, the depth sensor is an incision device 214 with an attached depth sensor tracking array 216. The tip of the incision device 214 can be calibrated with respect to the depth sensor tracking array 216 and tracked with respect to tracking system coordinates using techniques known in the art such as those described in U.S. Pat. No. 7,043,961. The depth sensor may also be a tracked computer-assisted surgical device such as the ones described above. The working tool attached to the computer-assisted device may be for example a probe, scalpel, saw, drill bit, blade, lancet, electrocautery device, and the like.

In a specific embodiment of the invention, the depth sensor incision device 214 measures the depth of the incision. The tracking system 205 may then calculate the relative position in 3-D between the tip of the incision device 214 and the registered bone. From the registered surgical plan, each of the tissue layers and their relative positions from the bone is also known.

In a particular embodiment, with reference to FIG. 4, the depth sensor may be a laser distance measurement device 404. In this embodiment, the optical receivers 206′ and laser distance measurement device 404 are shown attached to a surgical light 402 in the operating room 400. It should be appreciated that the articulating lasers 210 may also be attached to the surgical light 402 and the tracking system computer 212 may be incorporated into/on surgical light 402.

The laser distance measurement device 404 may be in the line of sight of the incision path to measure the depth of the incision. The depth may be measured using time-of-flight algorithms. In a particular embodiment, the laser distance measurement device 404 may be a 2-D scanning, 3-D scanning, or raster scanning laser device. A scan of the incision may be created and the resulting image may be analyzed using topographical imaging software to determine the depth and/or a position of the incision during the surgical procedure. The topographical information may also be used to provide real-time depth information while the user is creating the incision on the subject as further described below.

FIG. 4 also depicts several other components in the setting of an operating room 400 that may aid in planning and/or executing the procedure. The operating room 400 generally includes a surgical device 406 and a computing system 408 having a planning computer 410 including a processor, the tracking computer 212 including a processor, a surgical device computer (not shown) including a processor, and peripheral devices. It is appreciated that processor functions are shared between computers, a remote server, a cloud computing facility, or combinations thereof. The planning computer 410, tracking computer 212, and device computer may be separate entities, or it is contemplated that their operations may be executed on just one or two computers. For example, the tracking computer 212 may also communicate and perform operations to control the surgical device 406. Likewise, the tracking computer may communicate with the controller that controls the articulating lasers 210. The peripheral devices allow the user to create the surgical plan and interface with the tracking system 205, articulating lasers 210, and surgical device 406 and may include: one or more user interfaces such as a monitor 412; and user-input mechanisms, such as a keyboard 414, mouse 416, pendent 418, joystick 420, foot pedal 422, or the monitor 412 may have touchscreen capabilities.

In a specific embodiment, the step of measuring the depth of the incision is measured using two or more lasers 210. The two or more lasers 210 are articulated or the beams therefrom rastered such that the laser projections or images overlap within the incision. As the tissue layers are incised and opened up, the intersection of laser beams that for example defines a line segment at skin level loses focus appears as separate lines as the tissue is cut to below the intersection for the lasers when the images are on deeper anatomy. A viewing camera, such as a high-definition video camera, monitors the amount the projections or images become out of focus. The system may be calibrated to accurately determine the depth of the incision based on the measured displacement between the projections or images captured by the viewing camera.

U.S. Pat. No. 4,939,709 in detailing an electronic visual display system for simulating the motion of a clock pendulum provides logic for selective light projection that is readily coupled with POSE or 3-D surgical zone data to indicate through light line projections onto tissue where addition tissue resection is needed according to a surgical plan.

The step of providing a signal prior to creating a second incision is then based on the measured depth of the incision and the registered surgical plan. The provided signal may come in a variety of different forms. In a particular embodiment, the lasers 210 are articulated to provide a new incision path on the particular tissue layer defined in the surgical plan. For example, the depth of the measured incision may indicate that the incision has passed through the skin layer 102 as shown in FIG. 1. The lasers 210 then articulate to project the incision path on the next tissue layer. The same methods described in FIGS. 3A-3D may also be used as the provided signal that updates accordingly as function of the measured tissue depth.

In a specific embodiment, the provided signal is given by a monitor in communication with the tracking system 205 or a computer-assisted surgical device. For example, the monitor may display the type of tissue and depth of the tissue as shown in FIG. 3D. The monitor may also display the 3-D model created in the surgical plan of each of the tissue layers. As the depth within the incision increases, the outer layers on the 3-D model may be subtracted, leaving the remaining tissues yet to be incised.

Embodiments of the present invention also provide a system and method for providing a real-time position and depth indicator to aid in creating an incision using one or more modulated pulsed articulating laser(s) 210 and a topographical imaging device 404. The topographical imaging device 404 may be for example an articulated 2D laser scanner, a 3D laser scanner, or a raster scanning system. The topographical imaging device 404 may be located on the surgical light 402, and calibrated with respect to the tracking system 205.

During an incision, the topographical imaging device 404 is constantly scanning the subject's anatomy. The resulting images are processed to determine, in real time, the position and depth of the incision. The position and depth may be compared to the data in the surgical plan. The pulsed laser(s) 210 can then be articulated to indicate the desired incision path and the pulses can be modulated to indicate the desired depth.

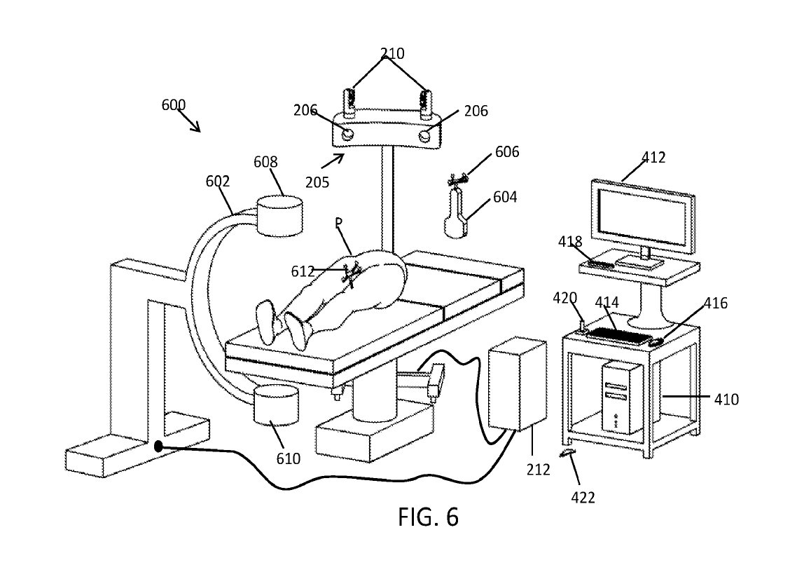

By way of example, FIGS. 5A-5C, illustrate the progression of an incision using one or more modulated pulsed articulating laser(s) 210 with a topographic imaging device 404. A general cube 500 is shown representing a subject's anatomy. The top surface 502 represents the subject's surface to be incised. First, the pulsed articulated laser(s) 210 is modulated at a rate undetectable by the human eye and articulated to indicate a solid incision path 504 as shown in FIG. 5A. Next, as the surgeon begins to create an incision 506, the images received from the topographical imaging device 404 are processed to determine the depth and position of the incision 506. The processed values are compared to the planned position and depth values. In an alternative embodiment, the topographical image itself may be correlated to any virtual incisions created in the surgical plan. In response, the light pulses are modulated such that a depth indication 508, such as line style change to a dashed line, dash length or a change in light frequency (color), is displayed as shown in FIG. 5B. Here, the dashes indicate the incision 506 requires further depth resection. As the surgeon approaches the desired depth, the dashes in specific embodiments may become less frequent, or the frequency of the pulses may become minimal. Once the desired depth is reached, no path is indicated in this region as shown in FIG. 5C. After the entire tissue layer has been incised to the proper depth, the process can be repeated on the next tissue layer until the target is reached. Therefore, the surgeon is provided an indication as to the desired path and depth of the incision in real-time. It is appreciated that the surface 502 can be an internal body tissue.

The example above may also be accomplished using one or more articulating laser(s) 210 with a continuous projection. Attached in front of the continuous projection may be a chopper to occlude the projection from the laser. The chopper may be articulated by an actuator controlled by a controller in communication with one of the computers described above to provide depth specific visual indicia to a surgeon. To create the same effect as the dashed depth indication 508, or frequency depth indication, the chopper is actuated or rotated to permit or occlude light accordingly depending on the measured parameters from the topographical imaging device.

Embodiments of the present invention also allow a surgeon to navigate to a target region through multiple fatty tissue layers. In subjects with a high BMI, the fatty tissue layers are constantly moving or being shifted throughout the incision. If a laser only highlights the position of the target region, the movement of the fatty tissue layers during the incision may result in an incision path away from the target region once the fatty tissue is normalized (i.e. the tissue is in its natural position without any external forces). Therefore, in a particular embodiment, the exterior surface of the subject's anatomy may also be tracked using one or more surface fiducial markers attached thereto. As the surgeon maneuvers the soft tissue during the incision, the tracking system 205 can track the relative changes between the exterior surface and the registered bone. The projections or images from the articulated lasers 210 can then articulate in accordance with the movement of the fatty tissue layers to provide an incision path directly to the target region regardless of how the exterior surface is handled by the surgeon during the incision. Thus, when the fatty tissue layers are normalized after the incision, the target region is exposed or accessible without any additional cuts that would otherwise be needed with prior art systems. It should be appreciated, that the use of a topographical imaging device may also account for the fatty tissue layers by constantly scanning the position, depth and even the width of the tissues during the incision. A combination of a topographical imaging device with the surface fiducial markers may provide additional information with regard to the exact position of the exterior surface of the skin, as well as the current depth of the incision in real-time.

Additionally, if the surgical procedure involves an articulating joint, such as the knee, tracked surface fiducial markers can be attached to the exterior surfaces of the operative bones (e.g., the skin of the femur and the tibia) to account for the articulation of the joint. If multiple image data set(s) of the subject in different POSEs were used in surgical planning, the position of the surface markers relative to one another can notify the system as to how much flexion or extension the knee is in and which data set should be used. Additionally, during the procedure, as the surgeon flexes and extends the knee, the tracking system 205 can measure a relative distance between the exterior surfaces and the bones. This depth information may also be used to adjust any relative measurements created in the surgical plan using ratios. For example, the surgical plan may have stored a measured relative distance from the bone to the skin to be approximately 6 mm. If the distance from the exterior surface fiducial marker and the registered bone is calculated as 5 mm, all of the other tissues relative to the bone may be reduced in the surgical plan by ⅚ths.

In a specific embodiment, the lasers 210 can be articulated to guide the surgeon in performing the surgical procedure on the targeted anatomical region once accessed through the incision. If the surgical plan, for example, includes the POSE of the bone cuts needed to receive implants to restore the mechanical axis of a subject's leg in total knee arthroplasty, the lasers 210 may be articulated to project an indication or image of the cuts to be made on the bone. Other applications include a projected outline for a craniotomy opening or the femoral head osteotomy in total hip arthroplasty.

With reference to FIG. 6, an operating room 600 having intra-operative imaging capabilities is shown. Intra-operative imaging may allow the surgeon to create a surgical plan on-the-fly, update the surgical plan, register the bone, or verify the surgical plan. The operating room 600 includes a fluoroscopy system 602 and an ultrasound probe 604 having an ultrasound tracking array 606. If fluoroscopy is used, the patient fiducial marker array 612 attached to the bone may include a set of radiopaque markers in a known geometric relationship with respect to a set of passive or active optical markers. The fluoroscopy system 602 may further include a tracking array or a set of fiducial markers to determine the location of the fluoro source 608 or fluoro detector 610.

In a specific embodiment, the surgeon may acquire a plurality of intra-operative images to create a desired incision path. The fluoro system 602 or ultrasound probe 604 can then register the incision path with respect to the bone. Subsequently, the articulating lasers 210 can provide incision positional and depth data as previously described.

In another embodiment, the ultrasound probe 604 is used to identify the exterior surface of the patient and measure the depth between the exterior surface and the bone. The depth measured by the ultrasound probe 604 is compared to the depth defined in the surgical plan to verify or update the depths defined in the surgical plan. Since the POSE of the bone and the ultrasound probe 604 are known by the tracking system 205, the probe 604 is easily swept along the patient's skin along the length of the desired incision path to verify/update the depths defined in the plan. Additionally, the ultrasound probe 604 or the fluoro system 602 (with or without contrasting agent depending on the application) may identify critical anatomy (i.e. nerves, arteries) to avoid. The user can then modify the incision path to avoid this critical anatomy. This is particularly helpful as some critical anatomy may have shifted if a pre-operative MRI or CT scan was used to create the surgical plan.

The methodology described herein can optimize a surgical approach with a minimally invasive procedure by adjusting the optimal surgical trajectory with lesser dissection and tissue damages via preserving the surgical access within relation to the tissue layers. One main advantage is that knowing the correct entry point for each tissue layer will allow the surgeon to normalize the skin tension prior to making an incision, so that the surgeon does not have to stretch the skin or create a larger opening when the entry position is missed and an adjustment relative to the bony anatomy is necessary.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

The foregoing description is illustrative of particular embodiments of the invention, but is not meant to be a limitation upon the practice thereof. The following claims, including all equivalents thereof, are intended to define the scope of the invention.

1. A method of planning a minimally invasive surgical incision in a subject, said method comprising:
receiving an image data set of an anatomical region of the subject;
creating a surgical plan within the image data set;
registering the anatomical region to the surgical plan;
articulating a laser to project a light indication on the subject indicative of a depth of a first incision.

2. The method of claim 1 wherein the receiving of an image of an anatomical region further comprises fusing a computed tomography (CT) image data set and a magnetic resonance imaging (MRI) image data set.

3. The method of claim 1 wherein the creating of the surgical plan within the image date set comprises:
segmenting a plurality of tissue types; and
measuring a distance between a bone and one or more tissue types of said plurality of tissue types.

4. The method of claim 1 wherein the registering the anatomical region to the surgical plan further comprises:
attaching a tracking array to a bone of the subject; and
registering the surgical plan to the bone using image-registration.

5. The method of claim 1 wherein the indication from the articulating laser is one of a focal point, an articulated drawn incision path, an image of an incision path, an outline of a tissue, an image of a tissue, or an image of text indicating a tissue and a depth of the tissue.

6. The method of claim 1 wherein the indication from the articulating laser is an image of text indicating a tissue type and a depth of the tissue type.

7. The method of claim 6 further comprising changing the text to a different tissue using an input mechanism.

8. The method of claim 1 wherein the creating a first incision on a subject further comprises using a computer-assisted surgical device, wherein the computer-assisted surgical device actively guides an incision device.

9. The method of claim 1 further comprising creating a first incision along the light indication.

10. The method of claim 9 further comprising:
measuring a depth of the first incision to yield a measured depth; and
providing prior to creating a second incision a signal is based on the measured depth and the surgical plan as a light projection on the subject.

11. The method of claim 1 further comprising collecting financial remuneration.

12. A system for implementing a surgical plan on a subject comprising:
a tracking array;
a processor receiving an initial positional input from said tracking array, three-dimensional scan data of a surgical field and comprising software for generating a surgical plan;
a laser positioned to project a light indication on the subject;
a controller controlling said laser to modify the light indications to indicate a preselected path and a preselected depth of incision, said articulating device receiving the position input and the surgical plan.

13. The system of claim 12 wherein said tracking array generates continuous positional inputs to said processor and said articulating device.

14. The system of claim 12 wherein said tracking array generates continuous positional inputs to said processor and said articulating device.

15. The system of claim 12 wherein said laser further comprises a chopper.

16. The system of claim 12 wherein said laser is pulsed.

17. The system of claim 12 further comprising a second laser having a light output aimed to intersect with the light indication.

18. The system of claim 12 wherein the light indication changes when within a preselected distance of from the preselected depth of the incision.

19. The system of claim 18 wherein the light indication change is one or more of color, intensity, or line style.
